(19) United States
(12) Patent Application Publication
(10) Pub. No.: US 2009/0000619 A1
(43) Pub. Date: Jan. 1, 2009

(54) Powder Inhaler, System for Opening and Emptying Capsules

(75) Inventors: Leon Gradon, Warsaw (PL); Arkadiusz Moskal, Warsaw (PL); Tomasz Sosnowski, Warsaw (PL)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: GLAXOSMITHKLINE PHARMACEUTICALS S.A., Poznan (PL)

(21) Appl. No.: 11/575,881

(22) PCT Filed: Sep. 8, 2005
(86) PCT No.: PCT/PL2005/000060

§ 371 Date: Aug. 14, 2008

(30) Foreign Application Priority Data
Sep. 23, 2004 (PL) P.370286

(51) Int. Cl. A61M 15/00

(52) U.S. Cl. 128/203.15

(57) ABSTRACT

The invention consists of a system for opening and emptying capsules in a powder inhaler for administering medications The inhaler according to the invention has a chamber with a system for opening the medication capsule in which the process of opening and emptying the medication capsule takes place as well as an aerodynamic chamber in which the medication particles are separated from the carrier capsules. The aerodynamic chamber may also be used as a mouthpiece.

The invention consists of a system for opening and emptying capsules in a powder inhaler for administering medications. Powder inhalers are used for administering specified amounts of pharmaceutically active substances or their mixtures in the form of finely disintegrated particles into the human bronchial tree.

Single and multi-dose powder inhalers are commonly used. These differ in terms of construction and operation. In known powder inhalers the active substance particles are drawn in by users while they breathe in.

The therapeutic effect of a medication depends on the amount of and the place where the active substance particles are deposited in the human bronchial tree. The smaller the active substance particles are the deeper they are deposited in the bronchial tree. The best effects are achieved when the size of the active substance particle does not exceed 5 μm. Such small particles, however, quickly agglomerate and cannot be stored for a longer time. In order to provide appropriate stability of the micronisation of the active substance its particles are set onto larger particles of pharmaceutically neutral substances, e.g. lactose particles. The connection between the small active substance particles and the lactose particle is stable during storage and therefore the particles of the medication do not agglomerate. The connection is, however, weak enough to quickly disintegrate due to the movement of air. During inhalation the subtle active substance particles are separated from the carrier particles. Thus prepared medication particles are stored in the form of single doses, usually in hard capsules. Multi-dose containers are also applied. For effective therapy it is important to achieve repeatable doses of the medication in successive inhalations.

In U.S. Pat. No. 3,991,761 and EP0005585 patent descriptions powder inhalers have been revealed with a chamber in which a capsule containing medication particles is inserted as well as an extended duct which is also used as a mouthpiece. The chamber with the capsule is separated from the air duct with a perforated partition. Before the process of inhaling begins the capsule with the medication is opened by piercing with needles which are located at the two semi-rounded ends of the capsule. The medication is released from the capsule as a result of its rotation caused by the flow of inhaled air. The particles of the medication which are set on the carrier particles are swept away by the air flow the speed of which depends on the force of inhaling. The disadvantage of these solutions is the incomplete emptying of capsules of the medication particles and, thus, the lack of repeated doses of the medication taken by the user. Additionally, the high resistance of air flow through the perforated partition make proper inhalation more difficult, particularly for elderly people whose breathing is not so efficient as well as for children.

In the British GB 2165159 patent description a powder inhaler was revealed in which a specified volume of medication is administered from a dispenser into the inhalation chamber by means of a specially shaped dial. The disadvantage of this solution is the unequal dosing of the medication since, depending on the sedimentation level of the powder, its apparent powder density changes and the successive doses which are measured in terms of volume vary in their contents of the medication.

The aim of the invention is developing an inhaler with a simple construction which enables the proper process of inhalation.

The inhaler according to the invention has a chamber with a system for opening the medication capsule in which the process of opening and emptying the medication capsule takes place as well as an aerodynamic chamber in which the medication particles are separated from the carrier capsules. The aerodynamic chamber may also be used as a mouthpiece.

In the chamber with the system for opening the medication capsule there is a capsule seat in which the medication capsule is placed as well as a piercing point, and a drive mechanism, particularly, a spring. The seat of the capsule is located above the inlet in the aerodynamic chamber.

It is advantageous when the longitudinal axis of the capsule is the same as the axis of the piercing device.

It is advantageous when the axis of the capsule is diagonal to the axis of the aerodynamic chamber.

Emptying the capsule takes place during its reciprocatory motion. While moving downwards the capsule is pierced with a piercing point the size of which is similar to the diameter of the capsule. It is then partially emptied of the medication. While moving upwards the capsule is further emptied of the medication. By pressing on the movable part of the dispenser chamber the capsule is moved in the direction of the piercing point and then, after it is pierced, the spring makes the capsule reverse and the powder it contains is emptied into the inhalation chamber. After the capsule rapidly stops in its reverse movement the medication particles which are left on the capsule walls fall off and fall into the aerodynamic chamber as a result of an inertia effect.

Between the chamber with the mechanism for opening capsules and the aerodynamic chamber there may be a perforated partition which makes it impossible for slivers of the capsule to get inside the latter chamber.

The piercing point may be of any shape, but it is the most advantageous when it is in the form of a pyramid and it is advantageous when the dimensions of its base are in the scope of 0.9-1.1 of the capsule diameter. After piercing the capsule the powder with the medication is emptied directly into the aerodynamic chamber.

In the inhaler, according to the invention, almost 100% of the medication is emptied from capsules and this guarantees reproducible doses of the inhaled medication.

Due to the air flow taking place during inhalation the medication particles are separated from the carrier particles in the aerodynamic chamber and are than inhaled by the patient.

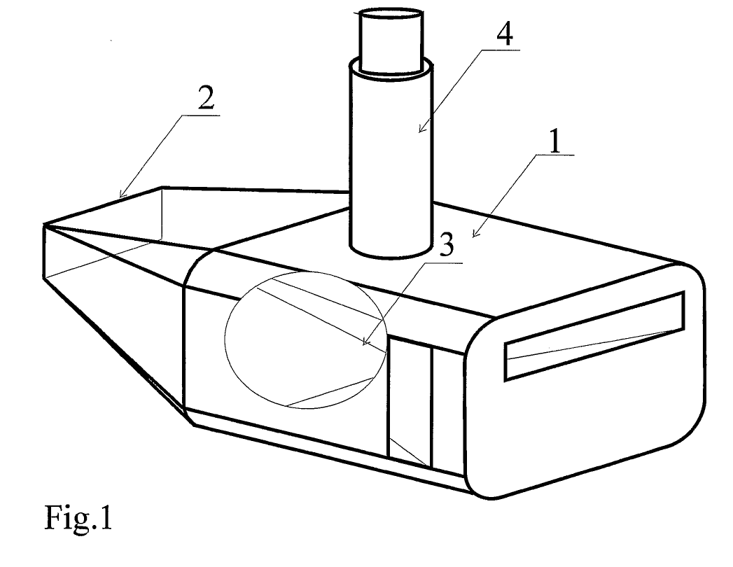

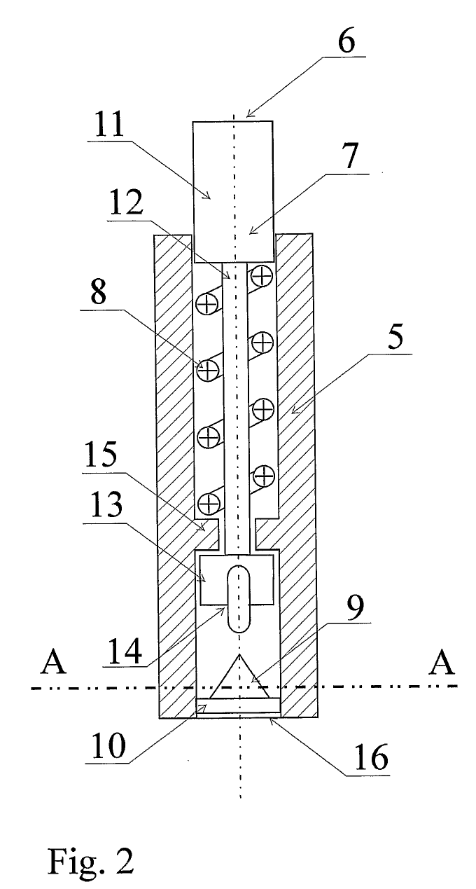

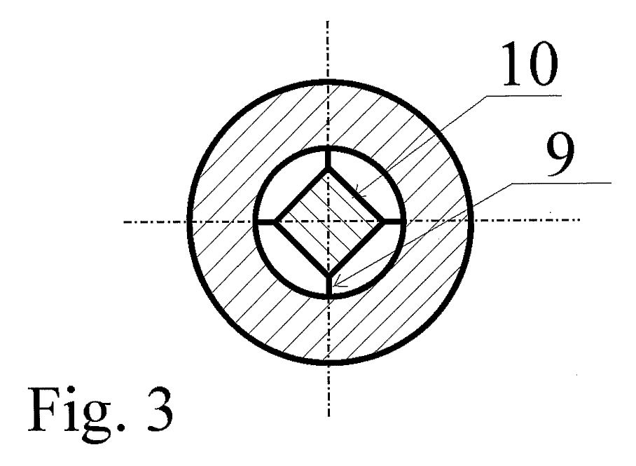

The object of the invention is depicted on a production example in the diagram in which FIG. 1 presents the inhaler, FIG. 2 presents a cross section of the system for emptying capsules and FIG. 3 presents a A-A plane section of the system for emptying capsules.

The inhaler consists of a body 1 with an aerodynamic chamber 3 and a mouthpiece 2 as well as a system for emptying capsules 4. The aerodynamic chamber inside the body is connected with the system for emptying capsules 4 through an opening.

The system for emptying capsules 4 consists of a fixed element 5 and a movable element 6. The fixed element 5 is cylinder-shaped and is permanently attached to the body 1. The movable element 6 comprises a piston 7 with a spring 8.

In the lower part of the fixed element 5 there is a piercing point 9. The piercing point 9 is set on perforated supports 10.

The piston 6 consists of a pusher 11, a piston rod 12 and a seat for placing capsules 13. The seat for placing capsules 13 has a semi-spherical cavity 14 the diameter of which is slightly smaller than the diameter of the capsules. On one side the spring 8 is set on an orifice 15 which is inside the fixed element 5 and on the lower edge of the pusher 11 on the other side.

Between the system for emptying capsules 4 and the aerodynamic chamber 3 there is a perforated partition 16 which makes it impossible for slivers of the capsule to get inside the aerodynamic chamber 3.

In order to empty a capsule it is necessary to remove the piston 7, place the capsule inside the cavity 14 of the capsule seat 13 and to replace the piston 7 in the fixed part. Pressing the pusher 11 with a finger makes the whole piston 7 move downwards and the piercing point 9 pierces the capsule. Releasing the pusher makes the piston 7 return to its original position and the powdered medication is emptied into the aerodynamic chamber. Stopping the piston rapidly during its reverse movement makes the medication particles which are left on the capsule walls fall off as a result of an inertia effect.

What is claimed is:

1. An inhaler for inhaling powdered medications with a system for emptying medication capsules and an aerodynamic chamber in which the inhalation powder is placed, wherein there is a system for emptying capsules 4 consisting of a fixed element in which a device for opening capsules is located, a spring, an orifice and a securing partition are located as well as a movable element in which a seat for placing capsules is located.

2. An inhaler according to claim 1, wherein the axis of the seat for placing capsules is the same as the axis of the device for opening capsules.

3. An inhaler according to claim 1, wherein the tool for opening capsules consists of a point the head of which is directed at the end of the capsule.

4. An inhaler according to claim 31 wherein the ratio of the piercing point basis dimension and the diameter of the pierced capsule is 0.9-1.1.

5. An inhaler according to claim 4, wherein the point which pierces capsules is in the form of a pyramid of any base or in the form of a cone.

6 An inhaler according to claim 1 wherein the return motion of the movable element is limited by the orifice.

7. An inhaler according to claim 1, wherein between the system for emptying capsules and the aerodynamic chamber there is a perforated partition.
